(19) United States
(12) Patent Application Publication
(10) Pub. No.: US 2013/0116776 A1
(43) Pub. Date: May 9, 2013

(54) EXTERNAL AORTIC RING AND SPOOL MECHANISM THEREFOR

(71) Applicant: Valtech Cardio, Ltd., Or Yehuda (IL)

(72) Inventors: Amir GROSS, Moshav Mazor (IL); Tal SHEPS, Givat Shmuel (IL); Eran MILLER, Moshav Beit Elazari (IL); Tal REICH, D.N. Gilboa (IL); Oz CABIRI, Macabim-Reut (IL)

(73) Assignee: VALTECH CARDIO, LTD., Or Yehuda (IL)

(21) Appl. No.: 13/666,141

(22) Filed: Nov. 1, 2012

Related U.S. Application Data
(60) Provisional application No. 61/555,575, filed on Nov. 4, 2011.

(51) Int. Cl. A61F 2/24

(52) U.S. Cl. CPC A61F 2/24
USPC 623/2.1

(57) ABSTRACT

A method for repairing an aortic valve of a heart of a patient is provided, the method comprising (1) placing around a portion of an aorta of the patient in a vicinity of the aortic valve, an adjustable implant structure comprising an adjusting mechanism coupled to a first portion of a flexible contraction member; and (2) adjusting a dimension of the aortic valve by adjusting a dimension of the implant structure by rotating a rotatable structure of the adjusting mechanism. Other embodiments are also described.

### CROSS-REFERENCES TO RELATED APPLICATIONS

The present application claims priority from U.S. Provisional Application 61/555,575 to Gross, filed on Nov. 4, 2011, which is incorporated herein by reference.

### FIELD OF THE INVENTION

The present invention relates in general to valve repair. More specifically, the present invention relates to repair of an aortic valve of a patient.

### BACKGROUND

Aortic insufficiency, also known as aortic regurgitation, is the leaking of the aortic valve of the heart that causes blood to flow in the reverse direction during ventricular diastole, from the aorta into the left ventricle. Aortic insufficiency can be due to abnormalities of either the aortic valve or the aortic root (the beginning of the aorta).

### SUMMARY OF THE INVENTION

In some embodiments of the present invention, apparatus is provided comprising an adjustable annuloplasty structure configured to repair a dilated aortic valve of a patient. At least a portion of the annuloplasty structure comprises a flexible, longitudinally-compressible element (e.g., coiled structures, stent struts, accordion structures, and/or a braided mesh). The annuloplasty structure is shaped to define a lumen thereof that houses a flexible member, e.g., a contracting wire. The annuloplasty structure comprises an adjusting mechanism which facilitates contracting and expanding of the annuloplasty structure. The adjusting mechanism comprises a rotatable structure (e.g., a spool) to which a first end portion of the flexible member is coupled. Typically, a second end portion of the flexible member is not coupled to the spool, but rather is coupled to a portion of the body portion of the annuloplasty structure. For some applications, the annuloplasty structure comprises a partial annuloplasty ring. For some applications, the annuloplasty structure comprises a full annuloplasty ring.

For some applications of the present invention, the annuloplasty structure is configured to be coupled (e.g., sutured, anchored, or otherwise coupled) directly to an external surface of a portion of the native aorta of the patient. For some applications of the present invention, the annuloplasty structure is coupled (e.g., sutured, anchored, or otherwise coupled) to a prosthetic aortic sparing device that is configured to be coupled to the external surface of a portion of the native aorta of the patient, and thereby, the annuloplasty structure is indirectly coupled to the native aorta. For some applications of the present invention, the annuloplasty structure is coupled (e.g., sutured, anchored, or otherwise coupled) to a prosthetic aortic graft that is configured to replace a portion of the native aorta and the aortic valve of the patient, and thereby, the annuloplasty structure is indirectly coupled to the native aorta.

As the operating physician rotates the spool of the adjusting mechanism in a first rotational direction, successive portions of the flexible member are wound around the spool. In response to continued rotation of the spool, increasing portions of the flexible member are wrapped around the spool, which causes the flexible member to pull on the second end of the elongate structure toward the adjusting mechanism. Responsively, the compressible element is compressed. Thus, the flexible member helps regulate a spatial configuration and adjust a perimeter of the annuloplasty structure in order to adjust a dimension of and repair the aortic valve.

There is therefore provided, in accordance with an application of the present invention, a method for repairing an aortic valve of a heart of a patient, including:

placing around a portion of an aorta of the patient in a vicinity of the aortic valve, an adjustable implant structure including an adjusting mechanism coupled to a first portion of a flexible contraction member; and

adjusting a dimension of the aortic valve by adjusting a dimension of the implant structure by rotating a rotatable structure of the adjusting mechanism.

In an application, adjusting the dimension of the aortic valve includes contracting the aortic valve.

In an application, adjusting the dimension of the aortic valve includes expanding the aortic valve.

In an application, the method further includes, subsequently to placing the adjustable implant structure, receiving information indicative of a function of the aortic valve of the patient, and adjusting the dimension of the implant structure includes adjusting the dimension of the implant structure at least in part responsively to the received information.

In an application, adjusting the dimension of the implant structure includes adjusting the dimension of the implant structure while the heart of the subject is beating.

In an application:

the adjustable implant structure includes a body portion, at least a first end thereof being coupled to the adjusting mechanism, the body portion being shaped to define a lumen therethrough, the contraction member being disposed within the lumen, and

placing the adjustable implant structure around the portion of the aorta includes placing the body portion around the portion of the aorta.

In an application, adjusting the dimension of the implant structure includes compressing at least a portion of the body portion.

In an application, a second portion of the flexible contraction member is coupled to a second end of the body portion, and adjusting the dimension of the implant structure includes reducing a length of a section of the flexible contraction member disposed between the second end of the body portion and the adjusting mechanism.

In an application, placing the body portion around the portion of the aorta includes placing around the portion of the aorta, a body portion that includes a coiled element surrounded by a braided mesh.

In an application, the method further includes unlocking a locking mechanism of the adjusting mechanism before rotating the rotatable structure.

In an application, unlocking the unlocking mechanism includes depressing a depressible portion of the unlocking mechanism, and the method further includes, subsequent to rotating the rotatable structure, locking the locking mechanism by releasing the depressible portion of the unlocking mechanism.

In an application:

the adjustable implant structure includes a first fastener at a first end of the adjustable implant structure, and a second fastener at a second end of the adjustable implant structure, and

placing the adjustable implant structure around the portion of the aorta includes coupling the first fastener to the second fastener.

In an application, coupling the first fastener to the second fastener includes passing a first portion of an elongate flexible member through a hole in the first fastener and through a hole in the second fastener.

There is further provided, in accordance with an application of the present invention, a method for use with an aortic valve of a heart of a patient, the aortic valve selected from the group consisting of: a native aortic valve and a prosthetic aortic valve, the method including:

coupling, to a portion of an aorta of the patient, a prosthetic tube, and an adjustable implant structure, disposed around a portion of the prosthetic tube, the adjustable implant structure including an adjusting mechanism, coupled to a first portion of a flexible contraction member, and

adjusting a dimension of the aortic valve of the patient by adjusting a dimension of the adjustable implant structure by rotating a rotatable structure of the adjusting mechanism.

In an application:

the prosthetic tube includes an inner wall and an outer wall,

the portion of the prosthetic tube includes a portion of the inner wall of the prosthetic tube, and

at least a portion of at least the flexible contraction member of the adjustable implant structure is disposed between the inner wall and the outer wall of the prosthetic tube.

In an application, coupling the prosthetic tube includes performing a valve-sparing aortic root replacement procedure.

In an application, the selected aortic valve includes the prosthetic aortic valve, and:

coupling the prosthetic tube includes coupling a prosthetic tube that includes the prosthetic aortic valve, and

adjusting the dimension of the aortic valve includes adjusting a dimension of the prosthetic aortic valve.

In an application, the selected aortic valve includes the native aortic valve, and adjusting the dimension of the aortic valve includes adjusting a dimension of the native aortic valve.

In an application, adjusting the dimension of the aortic valve includes contracting the aortic valve.

In an application, adjusting the dimension of the aortic valve includes expanding the aortic valve.

In an application, the method further includes, subsequently to coupling the prosthetic tube, receiving information indicative of a function of the aortic valve of the patient, and adjusting the dimension of the implant structure includes adjusting the dimension of the implant structure at least in part responsively to the received information.

In an application, adjusting the dimension of the implant structure includes adjusting the dimension of the implant structure while the heart of the subject is beating.

In an application, coupling the prosthetic tube and the adjustable implant structure includes coupling, to the portion of the aorta, the prosthetic tube and an adjustable implant structure that includes a body portion:

at least a first end of the body portion being coupled to the adjusting mechanism,

the body portion being disposed around the portion of the prosthetic tube, and shaped to define a lumen therethrough, the contraction member being disposed within the lumen.

In an application, adjusting the dimension of the implant structure includes compressing at least a portion of the body portion.

In an application, a second portion of the flexible contraction member is coupled to a second end of the body portion, and adjusting the dimension of the implant structure includes reducing a length of a section of the flexible contraction member disposed between the second end of the body portion and the adjusting mechanism.

In an application, the method further includes unlocking a locking mechanism of the adjusting mechanism before rotating the rotatable structure.

In an application, unlocking the unlocking mechanism includes depressing a depressible portion of the unlocking mechanism, and the method further includes, subsequent to rotating the rotatable structure, locking the locking mechanism by releasing the depressible portion of the unlocking mechanism.

There is further provided, in accordance with an application of the present invention, a method for use with an aortic valve of a heart of a patient, the method including adjusting a dimension of the aortic valve by rotating a rotatable structure of an adjusting mechanism of an implant structure that is coupled to an external surface of a portion of an aorta of the patient.

In an application, adjusting the dimension of the aortic valve includes contracting the aortic valve.

In an application, adjusting the dimension of the aortic valve includes expanding the aortic valve.

In an application, the method further includes receiving information indicative of a function of the aortic valve of the patient, and adjusting the dimension of the implant structure includes adjusting the dimension of the implant structure at least in part responsively to the received information.

In an application, adjusting the dimension of the implant structure includes adjusting the dimension of the implant structure while the heart of the subject is beating.

In an application, rotating the rotatable structure includes rotating a rotatable structure of an adjusting mechanism of an implant structure that includes a flexible contracting member that is disposed around the portion of the aorta of the patient.

In an application, the contracting member has a first end portion that is coupled to the adjusting mechanism, a second end portion, and section between the second end portion and the adjusting mechanism that has a length, and adjusting the dimension of the aortic valve by rotating the rotatable structure includes adjusting the length of the section of the contracting member, by rotating the rotatable structure.

In an application, the method further includes unlocking a locking mechanism of the adjusting mechanism before rotating the rotatable structure.

In an application, unlocking the unlocking mechanism includes depressing a depressible portion of the unlocking mechanism, and the method further includes, subsequent to rotating the rotatable structure, locking the locking mechanism by releasing the depressible portion of the unlocking mechanism.

The present invention will be more fully understood from the following detailed description of embodiments thereof, taken together with the drawings, in which:

### BRIEF DESCRIPTION OF THE DRAWINGS

FIG. 1 is a schematic illustration of an annuloplasty structure surrounding a portion of a native aortic valve of a patient, in accordance with some applications of the present invention;

FIG. 2 is schematic illustration two annuloplasty structures surrounding respective portions of the native aortic valve, in accordance with some applications of the present invention;

FIG. 3 is a schematic illustration of two partial annuloplasty ring structures surrounding a prosthetic tubular structure configured to be coupled to the aorta, in accordance with some applications of the present invention;

FIGS. 4 and 5 are schematic illustrations of a partial annuloplasty ring structure surrounding a prosthetic tubular structure configured to be coupled to the aorta, in accordance with respective applications of the present invention;

FIG. 6 is a schematic illustration of two full annuloplasty ring structures surrounding respective portions of a prosthetic tubular structure configured to be coupled to the aorta, in accordance with respective applications of the present invention;

FIGS. 7 and 8 are schematic illustrations of a full annuloplasty ring structure surrounding a prosthetic tubular structure configured to be coupled to the aorta, in accordance with respective applications of the present invention;

FIG. 9 is a schematic illustration of an adjusting mechanism coupled to the annuloplasty structures described herein, in accordance with some applications of the present invention;

FIGS. 10 and 11 are schematic illustrations of the full and partial annuloplasty ring structures, in accordance with some applications of the present invention;

FIG. 12 is a schematic illustration of a contracting member coupled to the annuloplasty structure, in accordance with some applications of the present invention;

FIG. 13 is a schematic illustration of a full annuloplasty ring structure surrounding a prosthetic tubular structure configured to be coupled to the aorta, in accordance with some applications of the present invention; and

FIG. 14 is a schematic illustration of a system for providing information indicative of heart function of the patient, and for facilitating adjusting a dimension of an annuloplasty ring structure in response to the information, in accordance with some applications of the invention.

### DETAILED DESCRIPTION OF EMBODIMENTS

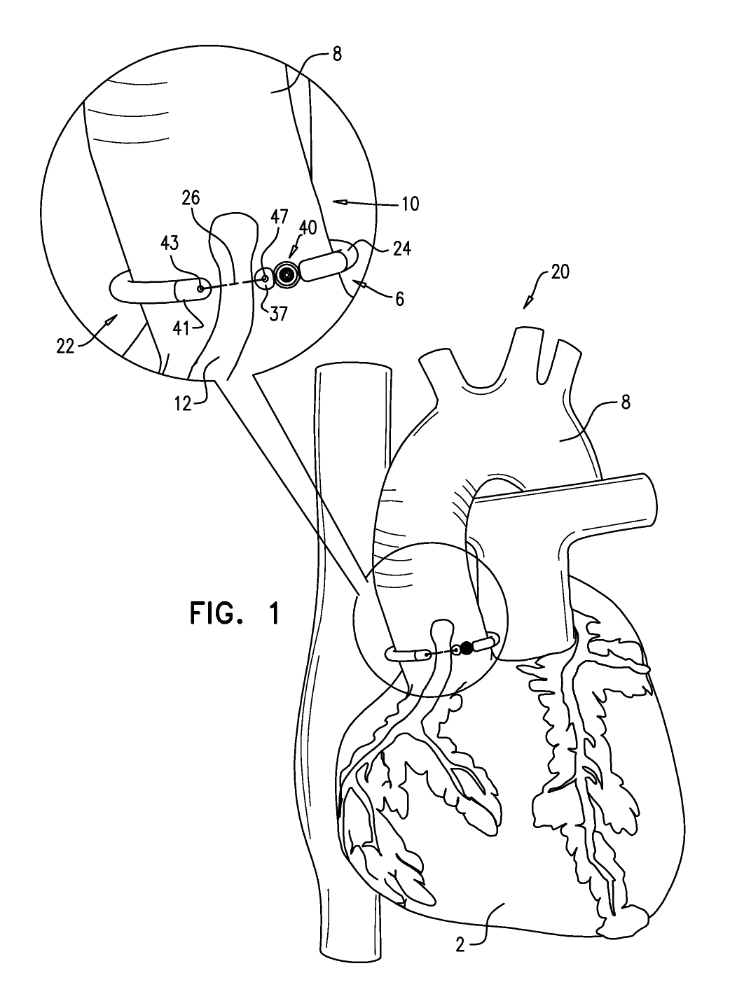

Reference is now made to FIG. 1, which is a schematic illustration of a system 20 for repairing an aortic valve 10 of a heart 2 of a patient, which comprises an adjustable annuloplasty ring structure comprising an adjustable partial annuloplasty ring structure 22 comprising an adjusting mechanism 40, in accordance with some applications of the present invention. Structure 22 is shaped so as to define first and second ends and a compressible body portion 24 between the first and second ends. Structure 22 is configured to surround a portion of a native aorta 8 in a vicinity of aortic valve 10 and to be coupled thereto by sutures, anchors, and/or any other suitable tissue-coupling element. A dimension of structure 22 is adjusted using adjusting mechanism 40 in order to adjust a dimension of valve 10.

Typically, as shown, structure 22 is configured to be coupled to an external surface of a portion of aorta 8 in a vicinity of aortic valve 10 (thereby, structure 22 defines an external annuloplasty structure). As shown by way of illustration and not limitation, structure 22 is coupled at a ventriculo-arterial junction 6 and is configured to remodel valve 10 and stabilize junction 6 during the remodeling. A portion of structure 22 is configured to pass under a coronary artery 12, as shown. Typically, structure 22 is configured to adjust dimensions of valve 10 to adjust a degree of coaptation of the leaflets of the aortic valve in order to remodel the valve.

It is to be noted that the scope of the present invention includes coupling structure 22 to aorta 8 at a sinotubular junction (shown as reference number 4 herein). For patients having a distended sinotubular junction 4 (i.e., the leaflets do not coapt properly), structure 22 can be adjusted to contract junction 4 (and thereby valve 10) and to facilitate coaptation of the leaflets (in addition to or in combination with a structure 22 implanted at ventriculo-arterial junction 6). For patients having a constricted sinotubular junction 4 (i.e., the leaflets prolapse), structure 22 can be adjusted to expand junction 4 (and thereby valve 10) and repair the prolapse.

Structure 22 is shaped so as to define first and second fasteners 41 and 37 at respective first and second ends of structure 22. First fastener 41 is shaped to define a hole 43 for passage therethrough of a first portion of an elongate flexible member 26 (e.g., a suture). The second portion of flexible member 26 is coupled to second fastener 37 that is shaped to define a hole 47 for passage therethrough of the second portion of flexible member 26. Flexible member 26 is configured to provide additional coupling of structure 22 to aorta 8.

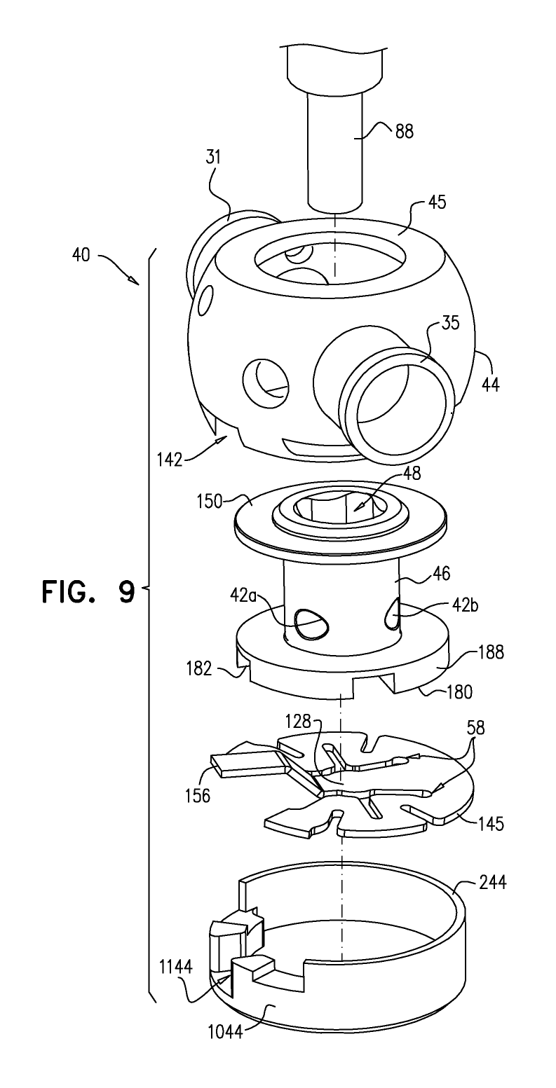

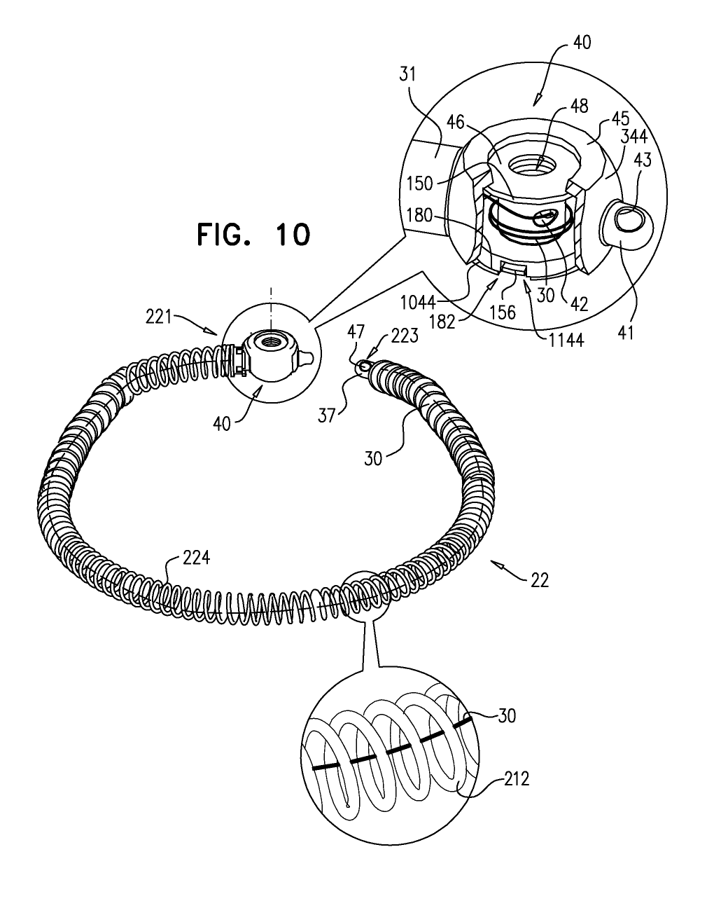

Reference is now made to FIGS. 1 and 9-10, which are schematic illustrations of adjusting mechanism 40 (FIG. 9), and structure 22 (FIG. 10), in accordance with some applications of the present invention. Structure 22 is shaped to define a flexible, tubular body portion 24 (FIG. 1) or 224 (FIG. 10) that is shaped so as to define a lumen along a longitudinal axis of structure 22 that houses at least one flexible longitudinal contracting member 30. At least a portion, e.g., the entirety, of body portion 24 or 224 comprises a compressible material (e.g., a coiled element 212), as shown by way of illustration and not limitation. For example, body portion 24 or 224 may comprise stent-like struts, or a braided mesh (independently of coiled element 212). Typically, coiled element 212 is surrounded by a braided mesh (not shown in FIG. 10 for clarity of illustration).

Typically, body portion 24 or 224 comprises a flexible biocompatible material, e.g., nitinol, stainless steel, platinum iridium, titanium, expanded polytetrafluoroethylene (ePTFE), or cobalt chrome. In some applications of the present invention, body portion 24 or 224 is coated with PTFE (Polytetrafluoroethylene). In other applications of the present invention, body portion 24 or 224 comprises accordion-like compressible structures which facilitate compression of the body portion, and thereby compression of the aorta and/or aortic valve when structure 22 is contracted. Body portion 24 or 224, when compressed, e.g., typically along a longitudinal axis of structure 22, enables portions of structure 22 to contract and independently conform to the configuration of the aorta. Thus, the compressible element of body portion 24 or 224 facilitates contraction of aortic valve 10 in response to contraction of structure 22.

Structure 22 comprises adjusting mechanism 40 disposed within a housing 344 and coupled to contracting member 30 (as described hereinbelow with reference to FIG. 9). Adjusting mechanism 40 is configured to adjust a degree of tension of contracting member 30 in order to adjust a perimeter of structure 22. Housing 344 of adjustment mechanism 40 is shaped so as to define at least a first coupling member 31. Body portion 24 or 224 comprises first and second ends 221 and 223. First end 221 is coupled to housing 344 via coupling member 31, and thereby to adjusting mechanism 40. Thus, adjusting mechanism 40 is aligned with body portion 24 or 224 along the longitudinal axis thereof.

Flexible contracting member 30 comprises a wire, a ribbon, a rope, or a band, comprising a flexible metal. Flexible contracting member 30 is coupled at a first end portion thereof to adjusting mechanism 40, which is coupled to a first end 221 of body portion 24 or 224. A second end portion of flexible contracting member 30 is coupled to a second end 223 of body portion 24 or 224. Thereby, a section of contracting member 30 is disposed between second end 223 and adjusting mechanism 40. Adjusting mechanism 40 typically adjusts the perimeter of structure 22 by adjusting a length of that section of contracting member 30. Typically, during a resting state of structure 22, flexible contracting member 30 is disposed in parallel with the longitudinal axis of structure 22. That is, flexible contracting member 30, for some applications does not comprise a continuous band that runs through the entire lumen of the annuloplasty devices described herein, and flexible contracting member 30 has at least one free end portion.

Typically, flexible contracting member 30 comprises a wire, a cable, or a rope, and taken together with the compressible element of body portion 24 or 224 and the braided mesh surrounding body portion 24 or 224, imparts flexibility to the entire annuloplasty structure.

Typically, flexible contracting member 30 comprises a flexible and/or superelastic material, e.g., nitinol, polyester, stainless steel, or cobalt chrome, and is configured to reside chronically within structure 22. In some applications of the present invention, flexible contracting member 30 comprises a braided polyester suture (e.g., Ti-Cron™). In some applications of the present invention, flexible contracting member 30 is coated with polytetrafluoroethylene (PTFE). In some applications of the present invention, flexible contracting member 30 comprises a plurality of wires that are intertwined to form a rope structure.

Adjusting mechanism 40 comprises a rotatable structure, such as a spool 46. The rotatable structure is rotatable in first and second opposing rotational directions with respect to housing 344 so as to expand and contract the annuloplasty structure, respectively. Spool 46 has a cylindrical body that is disposed perpendicularly with respect to the longitudinal axis of structure 22. As shown in FIG. 9, spool 46 is shaped to provide at least one hole 42 for coupling of the first end portion of flexible contracting member 30 thereto and, thereby, to adjusting mechanism 40. For some applications of the present invention, spool 46 is shaped to define one or more holes 42 (e.g., a first hole 42a and a second hole 42b) configured for looping a portion of contracting member 30 therethrough, as described hereinbelow.

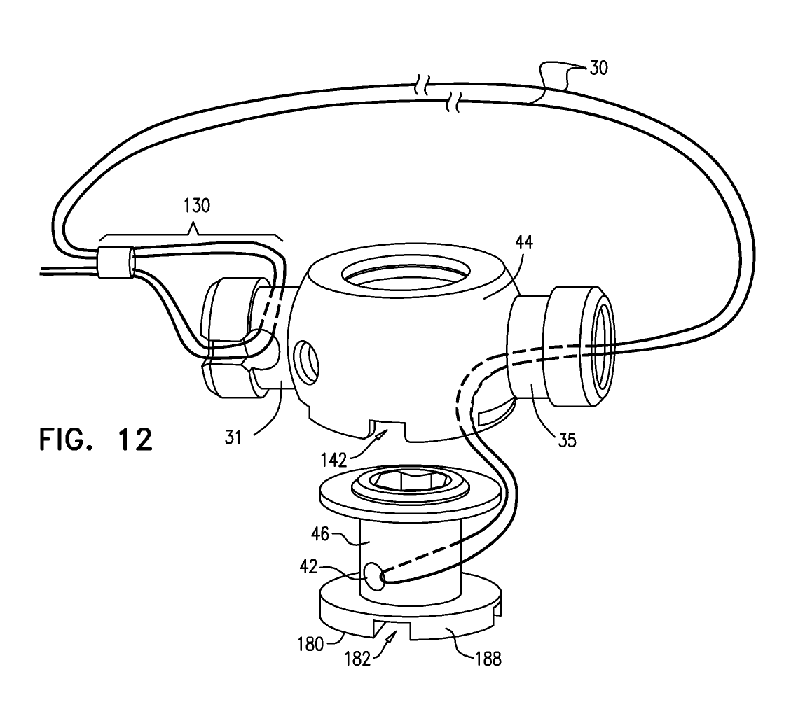

In some applications: (a) a middle portion, which defines the first end portion, of contracting member 30 is coupled to spool 46 by being looped through one or more holes 42, (b) first and second portions that extend from the first end portion looped through spool 46 extend from first end 221 toward second end 223 of structure body portion 24 or 224, and (c) first and second free ends of contracting member 30 are coupled to second end 223 of body portion 24 or 224 and define a second end portion of contracting member 30 (e.g., as described with reference to FIG. 12).

It is to be noted that housing 344 (and mechanism 40) may be disposed at any suitable location along structure 22. In some applications of the present invention, housing 344 may be disposed in the middle of the section of body portion 24 or 224 that is compressible. For some applications, a plurality of housings and adjusting mechanisms 40 described herein may be coupled to the annuloplasty structure. Each adjusting mechanism 40 may be coupled to a respective contracting member 30 which controls a respective portion of the annuloplasty structure.

Reference is now made to FIG. 9, which is a schematic illustration showing a relationship among individual components of adjusting mechanism 40, in accordance with some applications of the present invention. Adjusting mechanism 40 is shown as comprising spool housing 44 which defines an upper surface 45 and a recess 142 at a lower surface thereof. A spool 46 is configured to be disposed within housing 44 and defines an upper surface 150, a lower surface 180, and a cylindrical body portion disposed vertically between surfaces 150 and 180. The cylindrical body portion of spool 46 is shaped so as to define a channel which extends from a first opening at upper surface 150 to a second opening at lower surface 180. It is to be noted that housing 44 is shown by way of illustration and not limitation and that the housing surrounding spool 46 may comprise housing 344 as described hereinabove with reference to FIG. 10.

Lower surface 180 of spool 46 is shaped to define one or more (e.g., a plurality, as shown) of recesses 182 which define structural barrier portions 188 of lower surface 180. It is to be noted that any suitable number of recesses 182 may be provided, e.g., between 1 and 10 recesses. For some applications, recesses 182 are provided circumferentially with respect to lower surface 180 of spool 46.

Typically, spool 46 comprises a locking mechanism 145. For some applications, locking mechanism 145 is coupled, e.g., welded, at least in part to a lower surface of spool housing 44. Typically, locking mechanism 145 defines a mechanical element having a planar surface that defines slits 58. The surface of locking mechanism 145 may also be curved, and not planar. Locking mechanism 145 is shaped to provide a protrusion 156 which projects out of a plane defined by the planar surface of the mechanical element. The slits define a depressible portion 128 of locking mechanism 145 that is disposed in communication with and extends toward protrusion 156.

In a resting state of locking mechanism 145 (i.e., a locked state of spool 46), protrusion 156 is disposed within a recess 182 of spool 46. Additionally, in the locked state of spool 46, protrusion 156 is disposed within recess 142 of housing 44.

Depressible portion 128 is aligned with the opening at lower surface 180 of spool 46 and is moveable in response to a force applied thereto by a distal force applicator 88. That is, distal force applicator 88 is configured to be disposed within the channel of spool 46. A distal end of applicator 88 is configured to push on depressible portion 128 in order to move depressible portion 128 downward so as to disengage protrusion 156 from within a recess 182 of spool and to unlock spool 46 from locking mechanism 145.

It is to be noted that the planar, mechanical element of locking mechanism 145 is shown by way of illustration and not limitation and that any suitable mechanical element having or lacking a planar surface but shaped to define at least one protrusion may be used together with locking mechanism 145.

A cap 1044 is provided that is shaped so as to define a planar surface and an annular wall having an upper surface 244 that is coupled to, e.g., welded to, the lower surface of spool housing 44. The annular wall of cap 1044 is shaped so as to define a recessed portion 1144 of cap 1044 that is in alignment with recess 142 of spool housing 44. Locking mechanism 145 is disposed between lower surface 180 of spool 46 and the planar surface of cap 1044.

In an unlocked state of adjusting mechanism 40, protrusion 156 of locking mechanism 145 is disposed within recessed portion 1144 of cap 1044. In the unlocked state, force applicator 88 extends through spool 46 and pushes against depressible portion 128 of locking mechanism 145. The depressible portion is thus pressed downward, freeing protrusion 156 from within a recess 182 defined by structural barrier portions 188 of the lower portion of spool 46. Additionally, protrusion 156 is freed from within the recessed portion of spool housing 44. As a result, contracting mechanism 40 is unlocked, and spool 46 may be rotated with respect to spool housing 44.

Cap 1044 functions to restrict distal pushing of depressible portion 128 beyond a desired distance so as to inhibit deformation of locking mechanism 145. For applications in which adjusting mechanism 40 is implanted in heart tissue, cap 1044 also provides an interface between adjusting mechanism 40 and the heart tissue. This prevents interference of heart tissue on adjusting mechanism 40 during the locking and unlocking thereof. Additionally, cap 1044 prevents damage to heart tissue by depressible portion 128 as it is pushed downward.

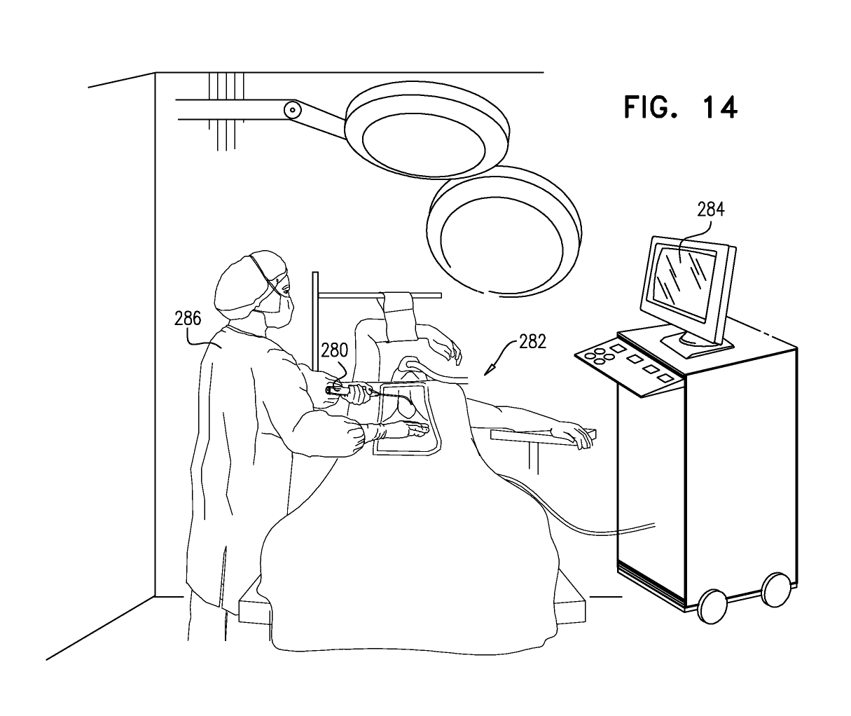

Spool 46 is shaped so as to define a driving interface 48. A rotation tool (not shown in FIG. 9, but, for example, shown as rotation tool 280 in FIG. 14) is configured to slide engage spool 46 at interface 48. The rotation tool is configured to rotate spool 46 by applying rotational force to spool 46 at interface 48. For some applications, a friction-reducing ring is disposed between upper surface 150 of spool 46 and upper surface 45 of spool housing 44.

For some applications the rotation tool used to rotate spool 46 may be shaped to provide force applicator 88, configured to unlock spool 46 from locking mechanism 145 (e.g., the force applicator is integral with the rotation tool). When unlocked, spool 46 may be bidirectionally rotated.

Following rotation of spool 46 such that contraction member 30 is contracted sufficiently to adjust the perimeter of the annuloplasty structure to a desired dimension so as to contract the annulus of the valve, spool 46 is then locked in place so as to restrict rotation of spool 46. Force applicator 88 is removed from within the channel of spool 46, releasing depressible portion 128, and thereby, depressible portion 128 returns to its resting state. As depressible portion 128 returns to its resting state, protrusion 156 is introduced within one of the plurality of recesses 182 of lower surface 180 of spool 46 and within recess 142 of housing 44, and thereby restricts rotation of spool 46.

It is to be noted that the contraction of the annuloplasty structures described herein is reversible. That is, rotating spool 46 in a second rotational direction that opposes the first rotational direction used to contract the annuloplasty structure, unwinds a portion of flexible contracting member 30 from around spool 46. Unwinding the portion of flexible contracting member 30 from around spool 46 thus feeds the portion of flexible contracting member 30 back into the lumen of body portion 24 or 224 of the annuloplasty structure, thereby slackening the remaining portion of flexible contracting member 30 that is disposed within the lumen of the body portion. Responsively, the annuloplasty structure gradually relaxes and expands (i.e., with respect to its contracted state prior to the unwinding) as the compressible element of body portion 24 gradually expands.

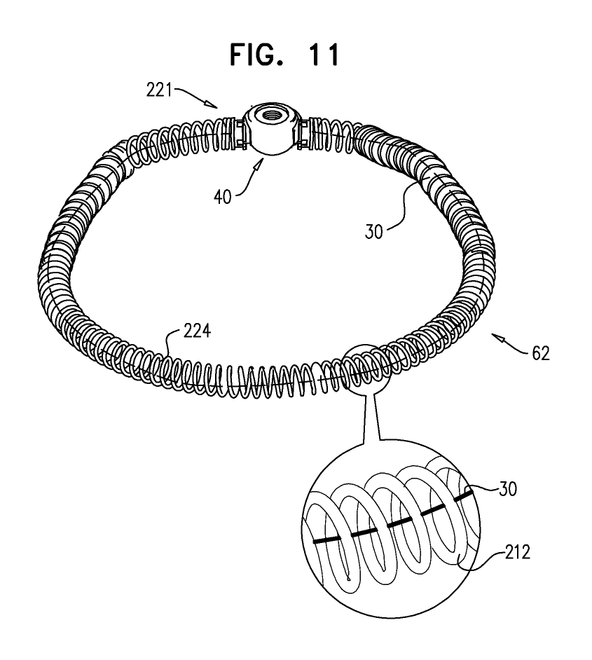

A second coupling member 35 of housing 44 is shown in FIG. 9 for embodiments in which a full annuloplasty ring is used to surround aorta 8 (FIG. 11). In such applications, first and second ends 221 and 223 of body portion 24 or 224 are coupled to coupling members 31 and 35 of housing 44 shown in FIG. 9. For applications in which a partial annuloplasty ring is used, housing 344 is used which is not shaped to define second coupling member 35 (e.g., as shown in FIG. 10).

Reference is now made to FIG. 11, which is a schematic illustration of an adjustable annuloplasty ring structure comprising an adjustable full annuloplasty ring structure 62 comprising an adjusting mechanism 40, in accordance with some applications of the present invention. Full annuloplasty ring structure 62 is similar to partial annuloplasty ring structure 22 described hereinabove with reference to FIGS. 1 and 10, with the exception that full annuloplasty ring structure 62 comprises housing 44 (described in FIG. 9) and the first and second ends of body portion 24 or 224 are coupled to housing 44 via coupling members 31 and 35, respectively. Structure 62 defines an external annuloplasty structure configured to surround a portion of aorta 8.

It is to be noted that for some applications of the present invention, flexible contracting member 30 of structure 62 may be coupled at both its first and second end portions, e.g., first and second ends, to spool 46 of adjusting mechanism 40. In some applications of the present invention, a first end of flexible contracting member 30 is coupled to spool 46 while a second end of flexible contracting member 30 is coupled to housing 44 which houses spool 46. For some applications, contracting member 30 comprises a continuous band that is looped through a portion of spool 46.

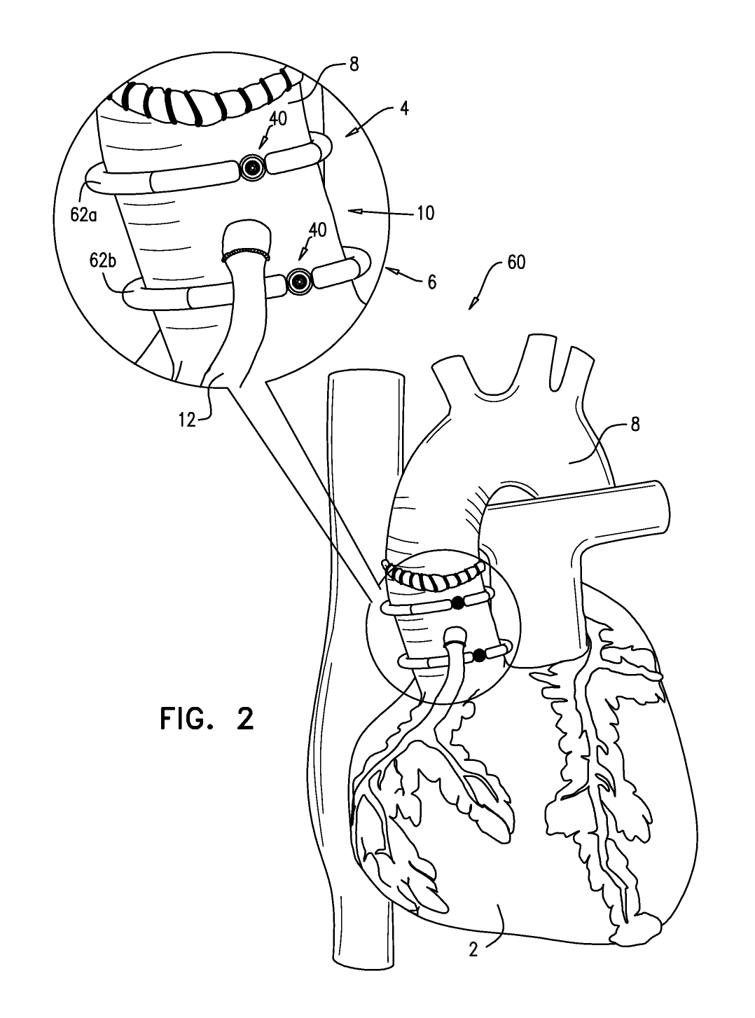

Reference is now made to FIG. 2, which is a schematic illustration of a system 60 for repairing aortic valve 10, comprising first and second full annuloplasty structures 62 (e.g., first annuloplasty structure 62a and second annuloplasty structure 62b), in accordance with some applications of the present invention. Structure 62 is described hereinabove with reference to FIGS. 9 and 11. Structures 62a and 62b are configured to be coupled directly to the external wall of aorta 8.

For such applications, structures 62a and 62b are configured to be expanded and contracted to adjust dimensions of aorta 8 and/or aortic valve 10, so as to adjust a degree of coaptation of the leaflets of the aortic valve. For patients having a distended sinotubular junction 4 (e.g., contributing to insufficient leaflet coaptation), structure 62a can be adjusted to contract junction 4 and to facilitate coaptation of the leaflets. For patients having a constricted sinotubular junction 4 (e.g., contributing to leaflet prolapse), structure 62a can be adjusted to expand junction 4 and to repair the prolapse. Structure 62b is configured to effect remodeling of valve 10 at the basal portion, at ventriculo-arterial junction 6.

It is to be noted that two structures 62a and 62b are shown by way of illustration and not limitation and that scope of the present invention includes implanting one structure 62 at either sinotubular junction 4 or ventriculo-arterial junction 6.

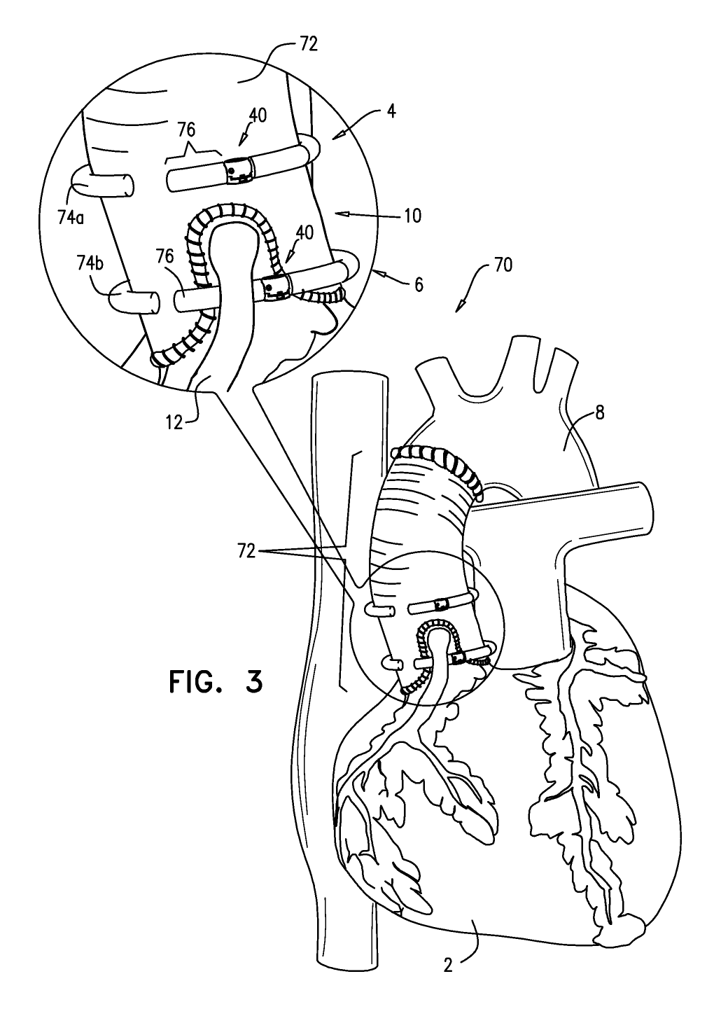

Reference is now made to FIG. 3, which is a schematic illustration of a system 70 for repairing aortic valve 10, comprising a prosthetic tube 72, in accordance with some applications of the present invention. Typically, prosthetic tube 72 comprises a prosthetic sparing tube for conducting a valve-sparing aortic root replacement in which a portion of the aortic root is replaced without replacement of the aortic valve.

As shown, first and second annuloplasty structures 74a and 74b are coupled to prosthetic tube surrounding respective portions of tube 72 that are equivalent to sinotubular junction 4 and ventriculo-arterial junction 6. For such applications structures 74a and 74b are configured to adjust dimensions of tube 72 at the commissural level and the basal level, respectively to adjust a degree of coaptation of the leaflets of aortic valve 10. Structures 74 define external annuloplasty structures configured to surround respective portion of aorta 8 via tube 72.

Structures 74a and 74b comprise partial annuloplasty structures, as described hereinabove with regard to structure 22 with reference to FIG. 1, with the exception that structures 74a and 74b each have respective extremities 76 which extend beyond respective adjusting mechanisms 40. It is to be noted that the scope of the present invention includes structures 74a and 74b comprising respective first and second fasteners 41 and 37 and flexible member 26, as described hereinabove with reference to FIG. 1.

For some applications, each of structures 74a and 74b comprises one or more contracting members 30. For some applications, each structure 74 comprises a first contracting member in a lumen of extremity 76 and a second contracting member in the lumen of the remaining body portion of structure 74. Respective first portions of the first and second contracting members are coupled to the spool of adjusting mechanism 40. Respective second ends of the first and second contracting members are coupled to respective free ends of structure 74. Alternatively, structure 74 comprises a single contracting member which passes through the spool of adjusting mechanism 40 and first and second ends of the contracting member are coupled to respective free ends of structure 74.

Contraction and expansion of structures 74a and 74b using adjusting mechanism 40 is described hereinabove with reference to FIGS. 1 and 9, mutatis mutandis. For such applications, structures 74a and 74b are configured to adjust dimensions of valve 10 to adjust a degree of coaptation of the leaflets of the aortic valve. For patients having a distended sinotubular junction 4 (i.e., the leaflets coapt insufficiently), structure 74a can be adjusted to contract junction 4 and to facilitate coaptation of the leaflets. For patients having a constricted sinotubular junction 4 (i.e., the leaflets prolapse), structure 74a can be adjusted to expand junction 6 and repair the prolapse. Structure 62b is configured to effect remodeling of valve 10 at the basal portion, at ventriculo-arterial junction 6.

Reference is now made to FIGS. 1-3. It is to be noted that structures 74a and 74b may be coupled directly to the external surface of aorta 8, as described hereinabove with regard to structures 22 and 62 with reference to FIGS. 1 and 2.

Reference is now made to FIGS. 1 and 3. It is to be further noted that structures 22 described with reference to FIG. 1 may be coupled to tube 72.

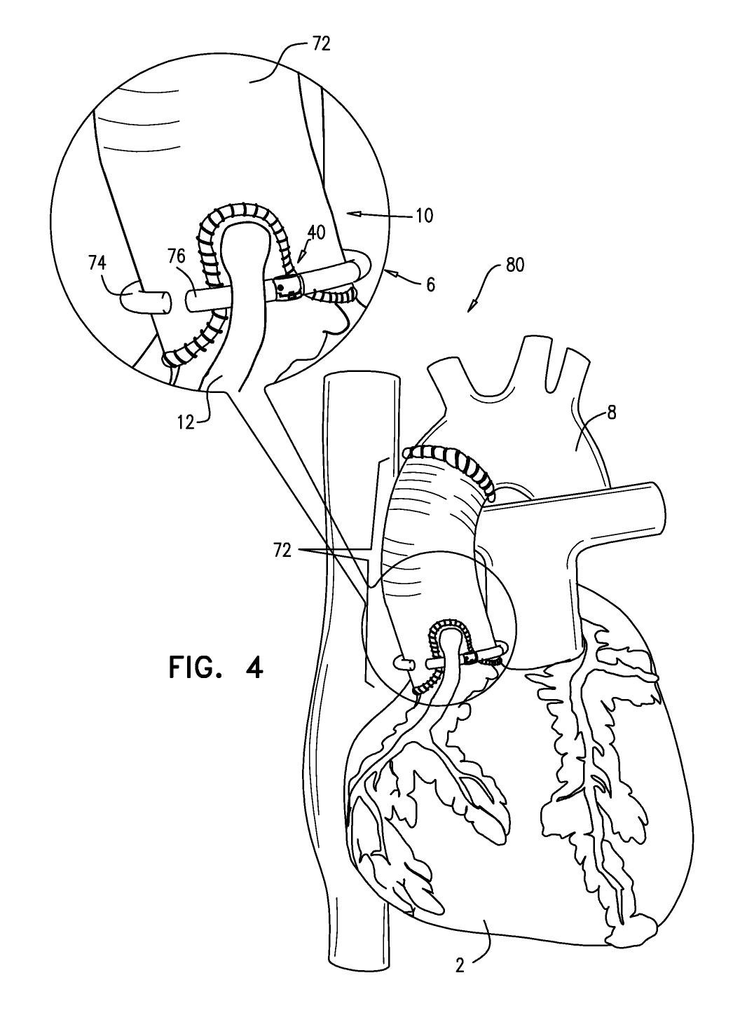

Reference is now made to FIG. 4, which is a schematic illustration of a system 80 for repairing aortic valve 10, as described hereinabove with reference to FIG. 3, with the exception that only one annuloplasty structure 74 is coupled to tube 72 in a portion thereof surrounding ventriculo-arterial junction 6.

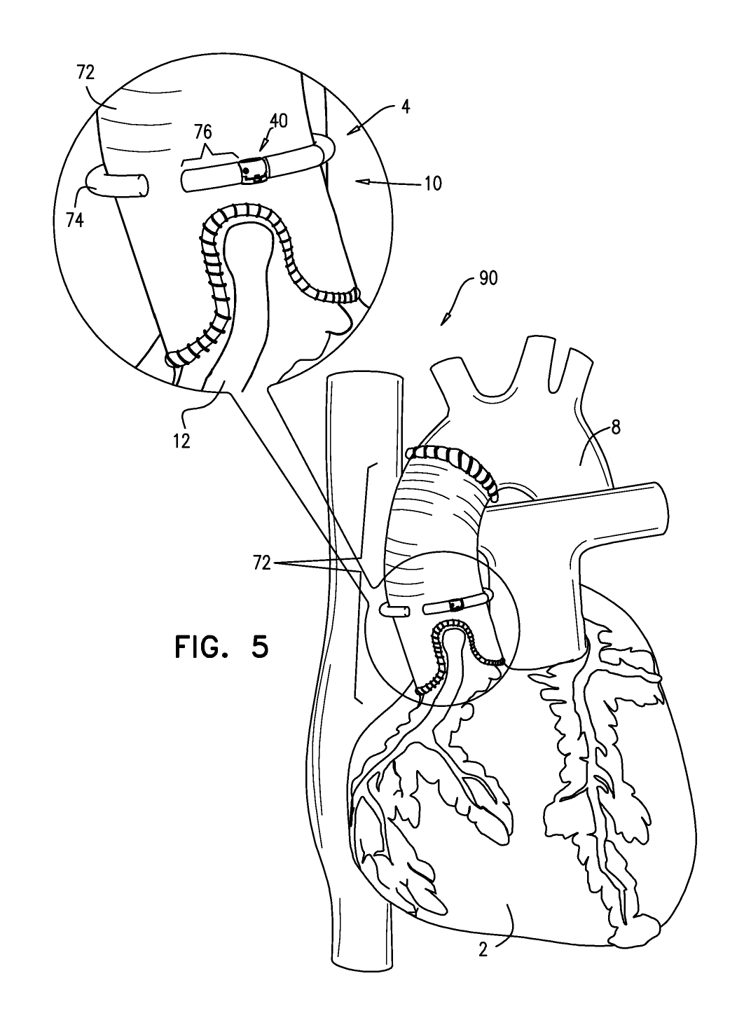

Reference is now made to FIG. 5, which is a schematic illustration of a system 90 for repairing aortic valve 10, as described hereinabove with reference to FIG. 3, with the exception that only one annuloplasty structure 74 is coupled to tube 72 in a portion thereof surrounding sinotubular junction 4.

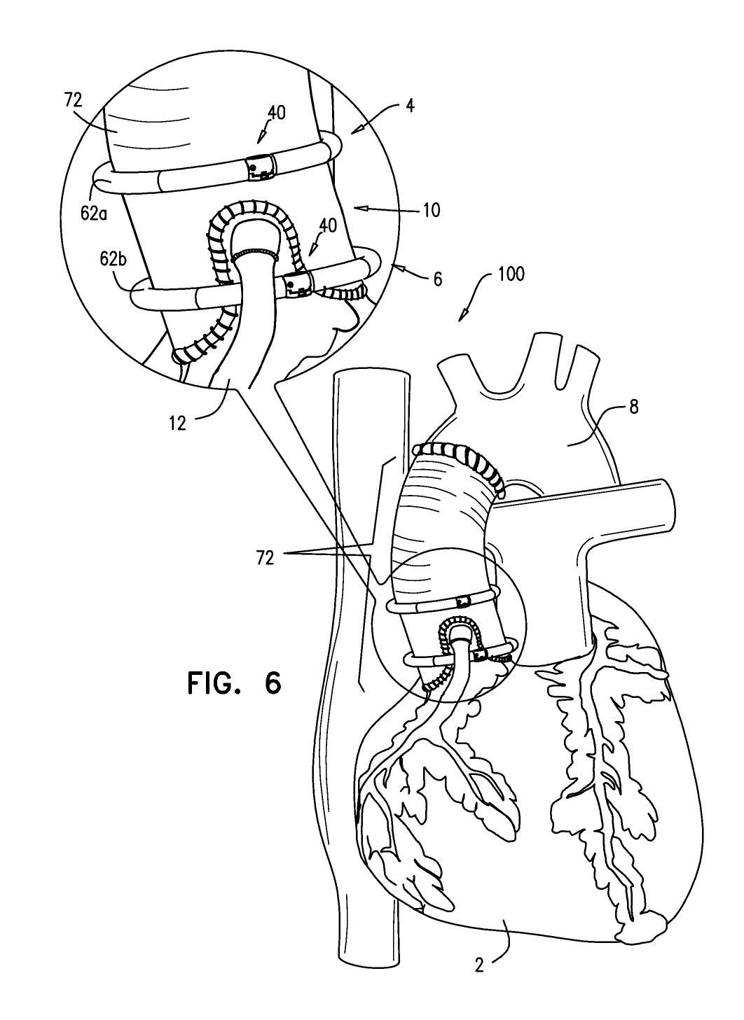

Reference is now made to FIG. 6, which is a schematic illustration of a system 100 for repairing aortic valve 10, as described hereinabove with reference to FIG. 3, with the exception that full annuloplasty structures 62a and 62b (as described hereinabove with reference to FIGS. 2, 9, and 11) are coupled to tube 72.

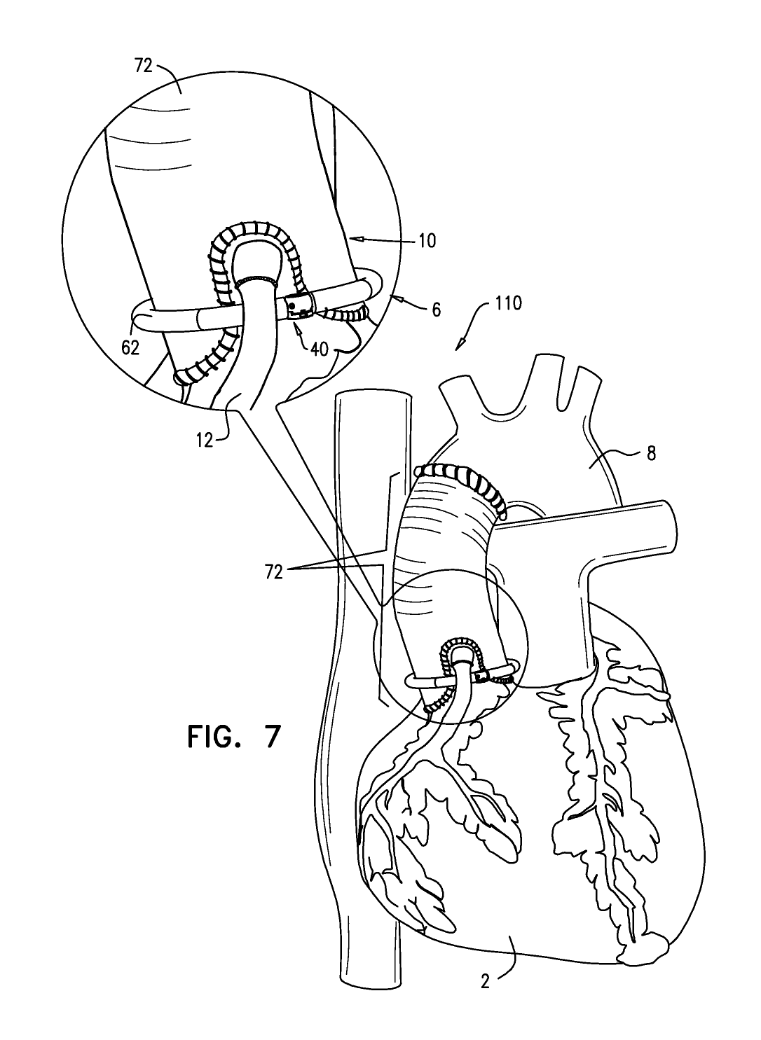

Reference is now made to FIG. 7, which is a schematic illustration of a system 10 for repairing aortic valve 10, as described hereinabove with reference to FIG. 6, with the exception that only one annuloplasty structure 62 is coupled to tube 72 in a portion thereof surrounding ventriculo-arterial junction 6.

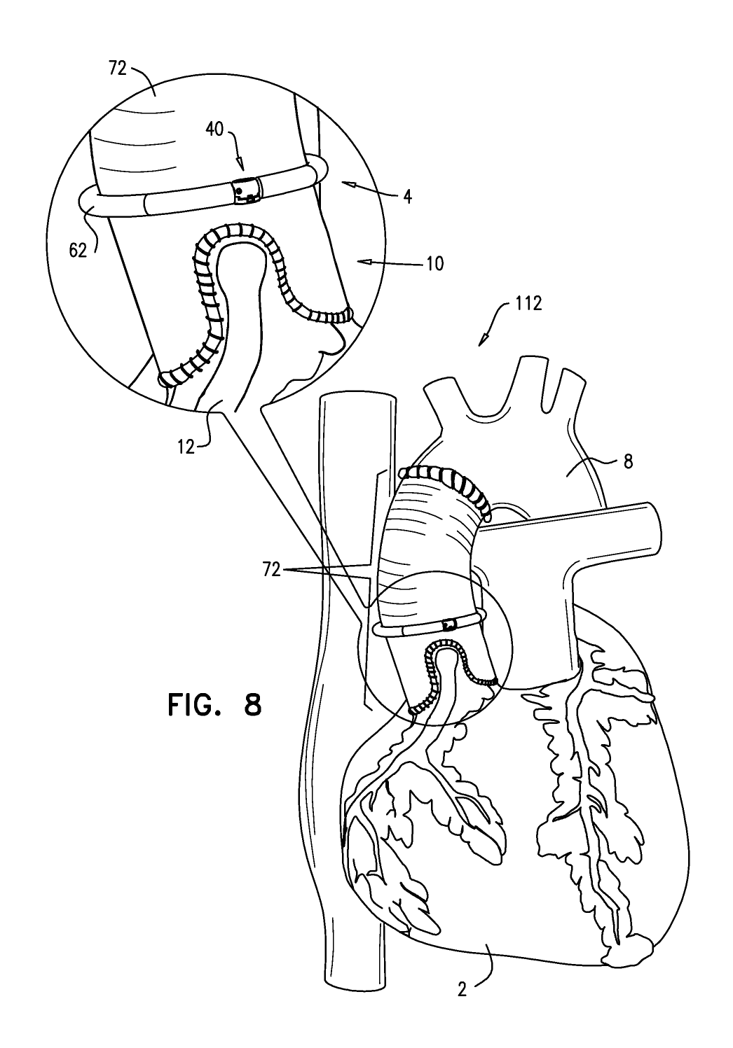

Reference is now made to FIG. 8, which is a schematic illustration of a system 112 for repairing aortic valve 10, as described hereinabove with reference to FIG. 3, with the exception that only one annuloplasty structure 62 is coupled to tube 72 in a portion thereof surrounding sinotubular junction 4.

FIG. 12 shows a relationship between contracting member 30, housing 44, and spool 46 for systems described herein comprising full annuloplasty ring structures 62 (e.g., described hereinabove with reference to FIGS. 6-8), in accordance with some applications of the present invention. Contracting member 30 is coupled to spool 46 by being looped through spool 46. Spool 46 is shaped to define one or more holes 42 configured for looping a portion of contracting member 30 therethrough. In such an application:

(a) a middle portion, which defines a first end portion, of contracting member 30 is coupled to spool 46 by being looped through one or more holes 42,

(b) first and second portions that extend (1) through coupling member 35 of housing 44, from the first end portion looped through spool 46 (2) through coupling member 31 of housing 44, and (3) toward a second end of the body portion of annuloplasty structure 62, and

(c) first and second free ends (and respective portions of contracting member 30) are coupled to the second end of the body portion of structure 62 and define a second end portion 130 of contracting member 30.

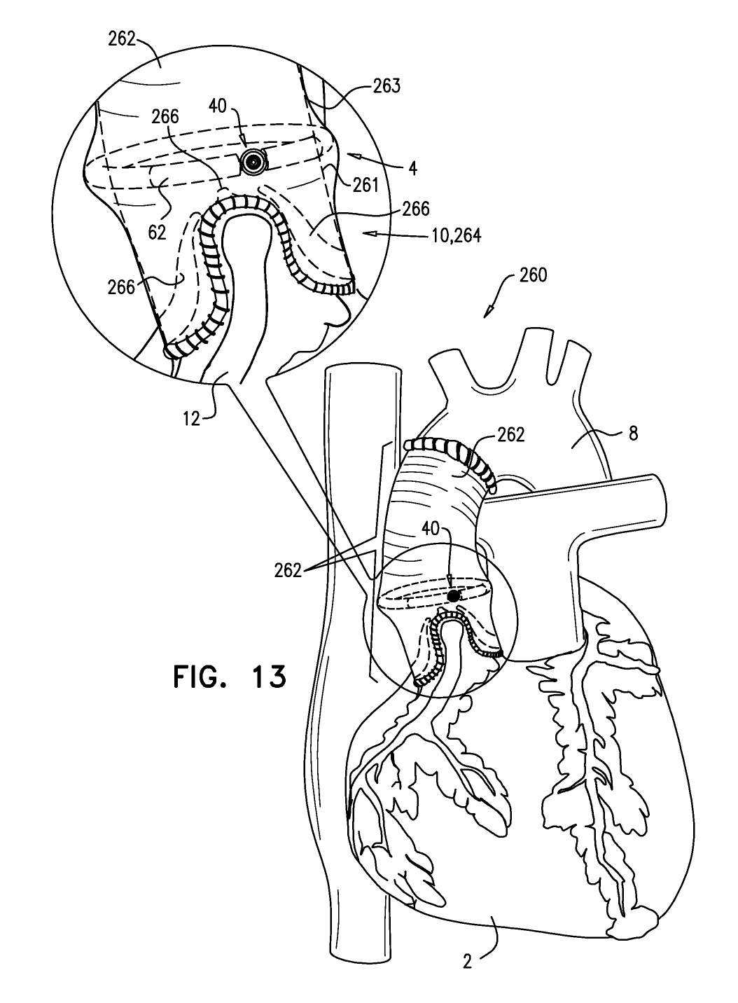

Reference is made to FIG. 13, which is a schematic illustration of a system 260, for repairing aortic valve 10, comprising a prosthetic tube 262, in accordance with some applications of the invention. System 260 comprises at least one lumen-adjusting structure, such as annuloplasty structure 62, and is generally as described hereinabove for system 112 with reference to FIG. 8, with the exception that the lumen-adjusting structure (e.g., annuloplasty structure 62) is integral with prosthetic tube 262 (e.g., embedded between an inner wall 261 and an outer wall 263 of the prosthetic tube). Typically, the lumen-adjusting structure is embedded within the prosthetic tube such that it is generally not exposed from the prosthetic tube, whereas adjusting mechanism 40 is typically exposed from the tube, thereby facilitating access thereto. It is to be noted that system 260 may comprise two or more lumen-adjusting structures (e.g., two or more annuloplasty structures), such as described for system 100 with reference to FIG. 6, mutatis mutandis.

As shown in FIG. 13, for some applications, prosthetic tube 262 comprises a prosthetic valve 264 (e.g., a xerographic or allogeneic valve), disposed within the prosthetic tube, and configured to replace the aortic valve. The prosthetic valve may comprise a tubular portion (e.g., a portion of prosthetic tube 262 and one or more leaflets 266). For such applications, the adjustment of the adjusting mechanisms adjusts a degree of coaptation of leaflets 266 of the prosthetic valve, in a manner similar to that described hereinabove for the adjustment of the degree of coaptation of leaflets of the native valve, mutatis mutandis. It is to be noted that any of the other prosthetic tubes described herein (e.g., with reference to FIGS. 3-8) may similarly comprise a prosthetic valve, mutatis mutandis.

Reference is made to FIG. 14. Following implantation of the annuloplasty structures described hereinabove, the dimensions of the annuloplasty structures may be adjusted by adjusting adjustment mechanism 40 thereof while the patient is not on a cardiopulmonary bypass pump (e.g., while the heart is beating). Adjustment (e.g., rotation) of the adjustment mechanisms off-pump facilitates adjustment while monitoring heart and/or valve function, and/or blood flow using imaging techniques, such as fluoroscopy and ultrasound (e.g., Doppler ultrasound), such that the physician may adjust until optimal heart function and/or blood flow is attained. For example, and as shown in FIG. 14, a rotation tool 280 may extend from the annuloplasty structure, to outside of the body of the subject 282, such that an operating physician 286 can adjust adjustment mechanism 40 of the annuloplasty structure while simultaneously and/or sequentially monitoring a display 284 that displays information indicative of the heart and/or valve function and/or the blood flow. It is to be noted that the scope of the invention includes other feedback systems, such as audio and/or tactile feedback, in addition to, or instead of, display 284.

Reference is again made to FIGS. 1-14. It is to be noted that the annuloplasty structures described herein are typically implanted using surgical techniques, such as incising and suturing, as considered appropriate by the operating physician. For example, and as shown in FIGS. 2, 6 and 7, for applications in which a full annuloplasty structure is implanted inferiorly to coronary artery 12, coronary artery 12 is typically cut and rejoined (e.g., by suturing), so as to facilitate such implantation.

Reference is again made to FIGS. 1-14. It is to be noted that any combination of annuloplasty structures described herein may be used to repair and remodel aortic valve 10. The combination of annuloplasty structures may be coupled to tube 72 or may be configured to be coupled directly to aorta 8.

Any of the annuloplasty structures described herein may be: (1) implanted directly on aorta 8, (2) implanted around the aorta when the aortic valve has been replaced with an aortic valve prosthesis, (3) used in combination with a valve-sparing aortic root replacement device, and/or (4) used in combination with a graft, as described hereinabove.

It is to be noted that systems 20, 60, 70, 80, 90, 100, 110, 120, and 260 for repairing a dilated annulus of the subject may be used to treat any cardiac valve of the subject, e.g., the aortic valve, the pulmonary valve, the mitral valve, and the tricuspid valve, mutatis mutandis.

It is to be still further noted that systems described herein for treatment of valves may be used to treat other annular muscles within the body of the patient, mutatis mutandis. For example, the systems described herein may be used in order to treat a sphincter muscle within a stomach of the subject, mutatis mutandis.

Additionally, the scope of the present invention includes applications described in one or more of the following:
U.S. patent application Ser. No. 12/435,291 to Maisano et al., entitled, “Adjustable repair chords and spool mechanism therefor,” filed on May 4, 2009, which published as U.S. Patent Application Publication 2010/0161041;
U.S. patent application Ser. No. 12/437,103 to Zipory et al., entitled, “Annuloplasty ring with intra-ring anchoring,” filed on May 7, 2009, which published as U.S. Patent Application Publication 2010/0286767;
U.S. patent application Ser. No. 12/548,991 to Maisano et al., entitled, “Implantation of repair chords in the heart,” filed on Aug. 27, 2009, which published as U.S. Patent Application Publication 2010/0161042;
PCT Patent Application PCT/IL2009/001209 to Cabiri et al., entitled, “Adjustable annuloplasty devices and mechanisms therefor,” filed on Dec. 22, 2009, which published as PCT Publication WO 10/073246;
PCT Patent Application PCT/IL2010/000357 to

Maisano et al., entitled, “Implantation of repair chords in the heart,” filed on May 4, 2010, which published as WO 10/128502; and/or
PCT Patent Application PCT/IL2010/000358 to Zipory et al., entitled, “Deployment techniques for annuloplasty ring and over-wire rotation tool,” filed on May 4, 2010, which published as WO 10/128503.

All of these applications are incorporated herein by reference. Techniques described herein can be practiced in combination with techniques described in one or more of these applications.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for repairing an aortic valve of a heart of a patient, comprising:
placing around a portion of an aorta of the patient in a vicinity of the aortic valve, an adjustable implant structure comprising an adjusting mechanism coupled to a first portion of a flexible contraction member; and
adjusting a dimension of the aortic valve by adjusting a dimension of the implant structure by rotating a rotatable structure of the adjusting mechanism.

2. The method according to claim 1, wherein adjusting the dimension of the aortic valve comprises contracting the aortic valve.

3. (canceled)

4. The method according to claim 1, further comprising, subsequently to placing the adjustable implant structure, receiving information indicative of a function of the aortic valve of the patient, and wherein adjusting the dimension of the implant structure comprises adjusting the dimension of the implant structure at least in part responsively to the received information.

5. The method according to claim 4, wherein adjusting the dimension of the implant structure comprises adjusting the dimension of the implant structure while the heart of the subject is beating.

6. The method according to claim 1, wherein:
the adjustable implant structure includes a body portion, at least a first end thereof being coupled to the adjusting mechanism, the body portion being shaped to define a lumen therethrough, the contraction member being disposed within the lumen, and
placing the adjustable implant structure around the portion of the aorta comprises placing the body portion around the portion of the aorta.

7. The method according to claim 6, wherein adjusting the dimension of the implant structure comprises compressing at least a portion of the body portion.

8. The method according to claim 6, wherein a second portion of the flexible contraction member is coupled to a second end of the body portion, and wherein adjusting the dimension of the implant structure comprises reducing a length of a section of the flexible contraction member disposed between the second end of the body portion and the adjusting mechanism.

9. The method according to claim 6, wherein placing the body portion around the portion of the aorta comprises placing around the portion of the aorta, a body portion that comprises a coiled element surrounded by a braided mesh.

10. The method according to claim 1, further comprising unlocking a locking mechanism of the adjusting mechanism before rotating the rotatable structure.

11. The method according to claim 10, wherein unlocking the unlocking mechanism comprises depressing a depressible portion of the unlocking mechanism, and wherein the method further comprises, subsequent to rotating the rotatable structure, locking the locking mechanism by releasing the depressible portion of the unlocking mechanism.

12. The method according to claim 1, wherein:
the adjustable implant structure includes a first fastener at a first end of the adjustable implant structure, and a second fastener at a second end of the adjustable implant structure, and
placing the adjustable implant structure around the portion of the aorta comprises coupling the first fastener to the second fastener.

13. The method according to claim 12, wherein coupling the first fastener to the second fastener comprises passing a first portion of an elongate flexible member through a hole in the first fastener and through a hole in the second fastener.

14.-27. (canceled)

28. A method for use with an aortic valve of a heart of a patient, the method comprising adjusting a dimension of the aortic valve by rotating a rotatable structure of an adjusting mechanism of an implant structure that is coupled to an external surface of a portion of an aorta of the patient.

29. The method according to claim 28, wherein adjusting the dimension of the aortic valve comprises contracting the aortic valve.

30. (canceled)

31. The method according to claim 28, further comprising receiving information indicative of a function of the aortic valve of the patient, and wherein adjusting the dimension of the implant structure comprises adjusting the dimension of the implant structure at least in part responsively to the received information.

32. The method according to claim 31, wherein adjusting the dimension of the implant structure comprises adjusting the dimension of the implant structure while the heart of the subject is beating.

33. The method according to claim 28, wherein rotating the rotatable structure comprises rotating a rotatable structure of an adjusting mechanism of an implant structure that includes a flexible contracting member that is disposed around the portion of the aorta of the patient.

34. The method according to claim 33, wherein the contracting member has a first end portion that is coupled to the adjusting mechanism, a second end portion, and section between the second end portion and the adjusting mechanism that has a length, and wherein adjusting the dimension of the aortic valve by rotating the rotatable structure comprises adjusting the length of the section of the contracting member, by rotating the rotatable structure.

35. The method according to claim 28, further comprising unlocking a locking mechanism of the adjusting mechanism before rotating the rotatable structure.

36. The method according to claim 35, wherein unlocking the unlocking mechanism comprises depressing a depressible portion of the unlocking mechanism, and wherein the method further comprises, subsequent to rotating the rotatable structure, locking the locking mechanism by releasing the depressible portion of the unlocking mechanism.
